(19) United States
(12) Patent Application Publication
(10) Pub. No.: US 2026/0256947 A1
(43) Pub. Date: Sep. 3, 2026

(54) COMBINATORY USE OF ADENO-ASSOCIATED VIRUSES AND FOCUSED ULTRASOUND FOR OVERCOMING THE BLOOD-BRAIN BARRIER

(71) Applicant: The Brigham and Women's Hospital, Inc., Boston, MA (US)

(72) Inventors: Fengfeng Bei, West Roxbury, MA (US); Nathan McDannold, Jamaica Plain, MA (US); Nicholas Todd, Needham, MA (US)

(21) Appl. No.: 19/489,184

(22) PCT Filed: May 31, 2024
(86) PCT No.: PCT/US2024/031935

§ 371 Date: Dec. 1, 2025

Related U.S. Application Data
(60) Provisional application No. 63/470,700, filed on Jun. 2, 2023.

(51) Int. Cl. A61K 48/00; A61K 9/00; A61K 41/00; C07K 14/005; C12N 15/86

(52) U.S. Cl. CPC A61K 48/005; A61K 9/0019; A61K 41/0028; A61K 48/0075; A61K 48/0083; C07K 14/005; C12N 15/86; C12N 2750/14122; C12N 2750/14143

(57) ABSTRACT

Provided herein are methods that use a combination of adeno-associated viruses (e.g., AAV.CPP.16 and AAV.CPP.21) and focused ultrasound for overcoming the blood-brain barrier, to deliver gene therapy reagents to the mammalian, e.g., human, brain.

### CLAIM OF PRIORITY

This application claims the benefit of U.S. Provisional Application Ser. No. 63/470,700, filed on Jun. 2, 2023. The entire contents of the foregoing are incorporated herein by reference.

### TECHNICAL FIELD

Provided herein are methods that use a combination of adeno-associated viruses and focused ultrasound for overcoming the blood-brain barrier, to deliver gene therapy reagents to the mammalian, e.g., human, brain.

### BACKGROUND

Genetic disorders impacting the central nervous system (CNS) pose a complex and significant problem within the United States healthcare system. Approximately 45% of the over 7,000 known rare genetic diseases are neurological disorders and 90% of genetic disorders seen in children have major neurological effects. This diverse group of disorders encompasses conditions such as Huntington's disease, spinocerebellar ataxias, and several lysosomal storage disorders, among others. While individually rare, together they affect millions of patients in the United States at all stages of life.

Genetic neurological disorders often carry a substantial disease burden. They significantly affect cognitive, emotional, and motor functions, leading to a marked reduction in the quality of life. These disorders often manifest early in life, leading to lifelong illness and substantial healthcare needs. Many conditions are progressive, ultimately leading to early death with treatment options limited to managing symptoms.

Significant advances have been made in recent years to identify the genetic mutations implicated in these disorders, many of which are now well characterized. A variety of nucleic acid therapeutics for targeting the genes of interest are also advancing rapidly¹. Methods for replacing lost gene function or silencing aberrant gene function are now well established in pre-clinical models. However, despite knowing the target and having therapeutics to correct the genetic defect, there is currently only one gene therapy that has FDA approval for a CNS disorder—Zolgensma, a gene replacement therapy used to treat children less than 2 years old with spinal muscular atrophy (SMA)².

A critical bottleneck preventing more therapeutics from achieving effective treatment in patients is the problem of delivering the therapy to the brain.

### SUMMARY

Provided herein are methods of delivering a transgene to a cell in the brain of a mammalian subject, comprising: using focused ultrasound-mediated disruption of the blood-brain barrier (FUS-BBB opening) in a selected region of the brain; and systemically administering to the subject an adeno-associated virus (AAV) comprising (i) a capsid protein comprising an amino acid sequence that comprises at least four contiguous amino acids from the sequence TVSALK (SEQ ID NO:2); TVSALFK (SEQ ID NO:3); KLASVT (SEQ ID NO:4); or KFLASVT (SEQ ID NO:5), and (ii) a transgene.

In some embodiments, the amino acid sequence comprises at least five contiguous amino acids from the sequence TVSALK (SEQ ID NO:4): TVSALFK (SEQ ID NO:8); KLASVT (SEQ ID NO:83); or KFLASVT (SEQ ID NO:84). In some embodiments, the amino acid sequence comprises at least six contiguous amino acids from the sequence TVSALK (SEQ ID NO:4); TVSALFK (SEQ ID NO:8); KLASVT (SEQ ID NO:83); or KFLASVT (SEQ ID NO:84).

In some embodiments, using FUS-BBB comprises: systemically administering a microbubble contrast agent, and applying focused ultrasound energy to the blood-brain barrier (FUS-BBB) in the selected region of the brain. In some embodiments, the microbubble contrast agent comprises perflutren lipid microspheres. In some embodiments, the focused ultrasound energy is applied at a frequency of from 200 kHz to 1.5 MHz, a burst duration of 10 ms, a number of bursts of from 30 to 180, a burst repetition frequency of from 0.25 to 2 Hz and a FUS Pressure of from 0.25 to 0.5 MPa.

In some embodiments, the subject is a human subject.

In some embodiments, the AAV is AAV9. In some embodiments, the AAV9 comprises AAV9 VP1. In some embodiments, the targeting sequence is inserted in a position corresponding to amino acids 588 and 589 of AAV9 VP1 comprising SEQ ID NO:85.

In some embodiments, the AAV is administered by parenteral delivery. In some embodiments, the parenteral delivery is via intravenous delivery. In some embodiments, the microbubble contrast agent and the AAV are delivered by intravenous delivery, optionally in a single composition.

In some embodiments, the transgene is listed in Table 2, optionally selected from the group consisting of mucolipin TRP cation channel 1 (MCOLN1); neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC), and aspartoacylase (ASPA); RNA interference; gene editing machineries. In some embodiments, the transgene is linked to sequences that promote expression of the transgene.

Also provided herein are adeno-associated viruses (AAVs) comprising (i) a capsid protein comprising an amino acid sequence that comprises at least four contiguous amino acids from the sequence TVSALK (SEQ ID NO:2); TVSALFK (SEQ ID NO:3); KLASVT (SEQ ID NO:4); or KFLASVT (SEQ ID NO:5), and (ii) a transgene, for use in a method of delivering a transgene to a cell in the brain of a mammalian subject, the method comprising: using focused ultrasound-mediated disruption of the blood-brain barrier (FUS-BBB opening) in a selected region of the brain; and systemically administering the AAV to the subject.

In some embodiments, the amino acid sequence comprises at least five contiguous amino acids from the sequence TVSALK (SEQ ID NO:4); TVSALFK (SEQ ID NO:8); KLASVT (SEQ ID NO:83); or KFLASVT (SEQ ID NO:84). In some embodiments, the amino acid sequence comprises at least six contiguous amino acids from the sequence TVSALK (SEQ ID NO:4); TVSALFK (SEQ ID NO:8); KLASVT (SEQ ID NO:83); or KFLASVT (SEQ ID NO:84).

In some embodiments, using FUS-BBB comprises: systemically administering a microbubble contrast agent, and applying focused ultrasound energy to the blood-brain barrier (FUS-BBB) in the selected region of the brain.

In some embodiments, the microbubble contrast agent comprises perflutren lipid microspheres. In some embodiments, the focused ultrasound energy is applied at a frequency of from 200 kHz to 1.5 MHz, a burst duration of 10 ms, a number of bursts of from 30 to 180, a burst repetition frequency of from 0.25 to 2 Hz and a FUS Pressure of from 0.25 to 0.5 MPa. In some embodiments, the subject is a human subject.

In some embodiments, the AAV is AAV9. In some embodiments, the AAV9 comprises AAV9 VP1. In some embodiments, the targeting sequence is inserted in a position corresponding to amino acids 588 and 589 of AAV9 VP1 comprising SEQ ID NO:85.

In some embodiments, the AAV is administered by parenteral delivery. In some embodiments, the parenteral delivery is via intravenous delivery. In some embodiments, the microbubble contrast agent and the AAV are delivered by intravenous delivery, optionally in a single composition.

In some embodiments, the transgene is listed in Table 2, optionally selected from the group consisting of mucolipin TRP cation channel 1 (MCOLNT); neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC), and aspartoacylase (ASPA). In some embodiments, the transgene is linked to sequences that promote expression of the transgene.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Results in mice using FUS+AAV9, FUS+AAV.PHPeB, FUS+AAV.CPP.16, and FUS+AAV.CPP.21. The white dots represent nuclear RFP signal. Ultrasound wave icons indicate areas to which FUS was applied. Adult BALB/C mice were used. AAVs expressing nuclear RFP under the ubiquitous promoter EF1a were administered via the tail vein immediately after FUS BBB opening. Animals were processed 3 weeks post-AAV-injection.

FIG. 2. A comparison of results in mice using AAV9 alone, FUS+AAV9, AAV.CPP.21 alone, or AAV.CPP.21+FUS, showing the significantly increased transduction achieved with the combination of AAV.CPP.21+FUS. Adult BALB/C mice were used. AAVs expressing nuclear RFP under the ubiquitous promoter EF1a were administered via the tail vein immediately after FUS BBB opening. Animals were processed 3 weeks post-AAV-injection.

FIGS. 3A-B. A, Images showing neuronal transduction in mice using FUS+AAV9, FUS+AAV.PHPeB, FUS+AAV CPP.16, and FUS+AAV CPP.21. The images show substantial overlap between neurons (NeuN) and RFP signal in the animals treated with FUS+AAV.CPP.16, and FUS+AAV.CPP.21. B) Quantification of percent neurons transduced in FUS-targeted (ultrasound) and non-targeted (control) hemispheres for cortex and striatum. Neuronal transduction levels reached over 60% in cortex and over 40% in striatum in the animals treated with FUS+AAV.CPP.16, and FUS+AAVCPP.21. Adult BALB/C mice were used. AAVs expressing nuclear RFP under the ubiquitous promoter EF1a were administered via the tail vein immediately after FUS BBB opening. Animals were processed 3 weeks post-AAV-injection.

FIGS. 4A-B. A) Images from mice administered differing doses of FUS+AAV.CPP.16; the left column shows contrast MRI images (ultrasound wave icons indicate areas to which FUS was applied), and the right column shows RFP expression. B) Quantification of RFP+ cells in mice treated as shown in 4A; n=3 in each condition. Adult C57BL/6 mice were used. AAVs expressing nuclear RFP under the ubiquitous promoter CAG were administered via the tail vein immediately after FUS BBB opening. Animals were processed 3 weeks post-AAV-injection.

### DETAILED DESCRIPTION

Delivery of AAV-based gene therapies directed to the CNS remains a critical challenge primarily due to the blood-brain barrier (BBB) which excludes essentially all large molecules (>−500 Da)³. Intravenous (IV), intrathecal (IT), intracerebroventricular (ICV), and intraparenchymal (IPa) injection have all been used as routes of administration in clinical trials for different disease and therapy combinations. Each has advantages, but each also has limitations that reduce clinical efficacy in some way.

The primary tradeoffs are around therapy concentration and brain coverage. In general, the less invasive methods of IV and IT injection can achieve more widespread distribution of the therapy over the whole brain but at the expense of significantly reduced therapy concentration in the brain compared to the more invasive methods of ICV and IPa injection. IV injections are very safe but rely on the vector being able to cross the BBB by itself, which is typically not the case for naturally occurring AAV serotypes. AAV9 has shown some CNS penetrance^{4,5} but can require such a high systemic dose to achieve sufficient concentration in the brain that it leads to toxicity in other organs⁶. A recent trial treating myotubular myopathy patients with an AAV9-based therapy given IV was halted after three patients in the high dose cohort died. Alternatively, IPa injection achieves the highest therapy distribution at the targeted site, with essentially no off-target toxicity^{7,8}. However, this requires a ~10 hr invasive brain surgery and coverage is limited to a small volume around the site of injection which may not be sufficient to adequately treated neurological disorders that affect the whole brain.

Major efforts are being put into engineering novel AAV capsids that can penetrate the BBB from systemic circulation⁹⁻¹¹. If successful, this approach could break the tradeoffs of conventional methods and achieve a combination of high therapy concentration and widespread brain coverage. However, significant challenges remain. Initial efforts in mice were found to be species-specific and not translatable to primates¹². Recent efforts in non-human primates are more promising but CNS-transducing efficiency is still low.

To treat the majority of neurological disorders that manifest in multiple brain regions, an approach to delivering AAV vectors that safely achieves a high percentage of neuronal transduction (>25%) over a large volume of the brain remains a critical unmet need.

To improve AAV delivery to the brain, described herein are methods that combine the technology of focused ultrasound-mediated disruption of the blood-brain barrier (FUS-BBB opening) with systemic administration of a novel AAV capsid, AAV.CPP, that has been engineered for improved BBB penetrance.

As shown herein, the combination of FUS-BBB opening with systemic administration of AAV.CPP achieves high levels of neuronal transduction not seen with alternative non-invasive approaches.

FUS-BBB opening combined with IV injection of AAV.CPP significantly increased the delivery of the gene therapy reagent to the brain compared to existing non-invasive methods published in the literature (and to FUS+AAV9 or administration of AAV.CPP alone). The enhancement seen in neuronal transduction when combining FUS-BBB opening with AAV.CPP, shown herein, was well above what was expected based on: 1) increases in neuronal transduction seen in going from AAV9 to AAV.CPP (without FUS); and 2) increases in neuronal transduction seen in using FUS in combination with AAV9.

An exemplary treatment paradigm would be to target BBB opening over large brain regions tailored to where the disease is most manifest. For example, bilateral targeting of the basal ganglia for the treatment of Huntington's disease or bilateral targeting of the putamen for Parkinson's disease. In some embodiments, the methods achieve >30% neuronal transduction at a sub-toxic systemic dose level. In areas outside of the FUS-targeted volume, the AAV.CPP vector will still provide a level of neuronal transduction significantly above what AAV9 can achieve. This level of neuronal transduction is expected to be sufficient to achieve a clinically relevant therapeutic effect.

The present methods can be applied to many different CNS genetic disorders as FUS can be targeted to one or more desired brain regions and AAV.CPP can package different gene therapy reagents for gene silencing or gene replacement. FUS-BBB opening is done without incisions or anesthesia, providing patients with a non-surgical option for gene therapy treatment superior to existing approaches that use IV injection of AAV vectors.

### FUS-BBB Opening

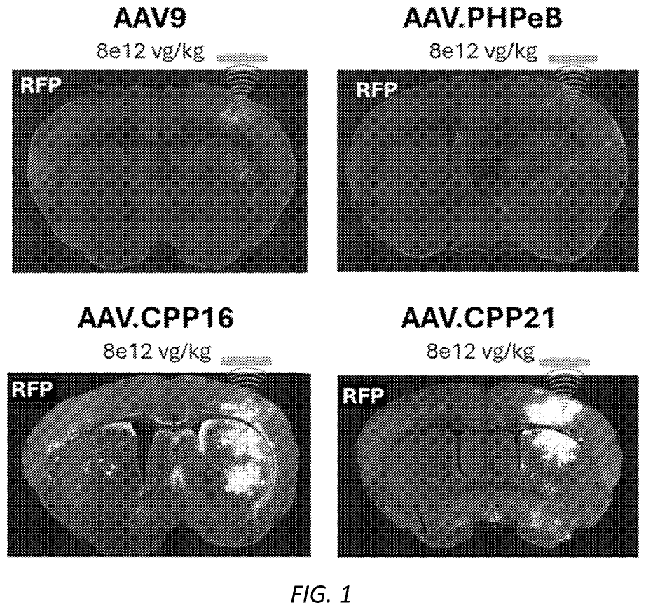

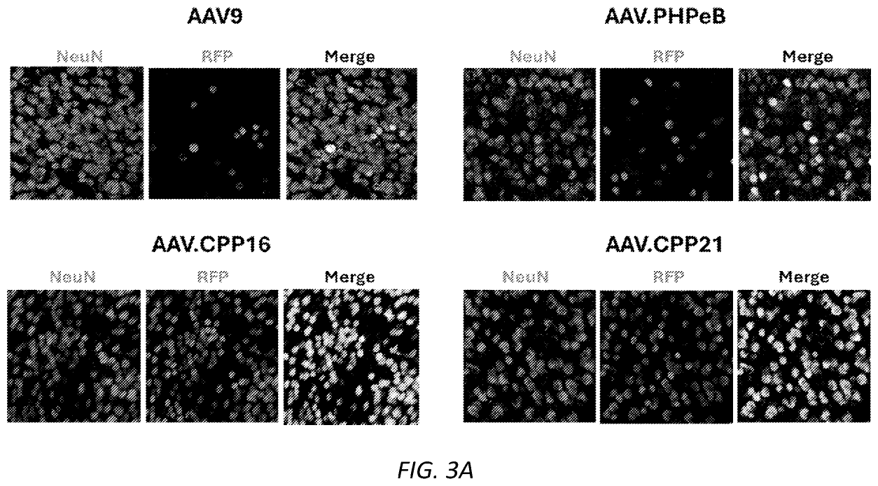

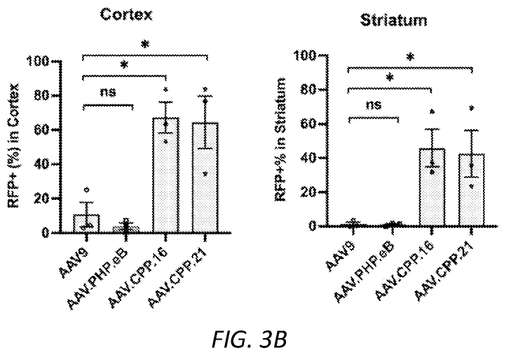

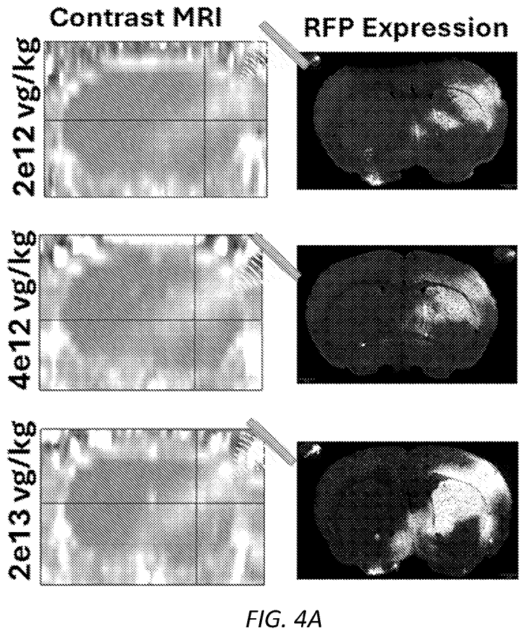

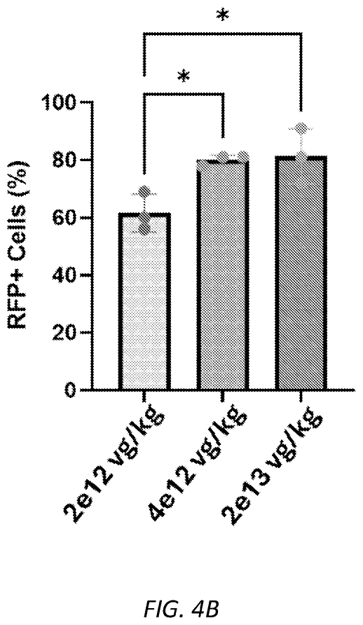

FUS-BBB opening achieves non-invasive and temporary disruption of the BBB by applying bursts of focused ultrasound energy through the intact skull in combination with systemically circulating microbubbles¹³. The FUS pressure field causes these gas-filled microbubbles to rapidly oscillate in size, creating forces on vessel walls that act to break tight junctions between endothelial cells and increase transcytosis across endothelial cells. The BBB re-closes over several hours¹⁴. Preclinical studies have demonstrated safe and effective brain delivery for a wide variety of normally non-penetrant agents such as chemotherapies (~500 Da)^{15 17}, antibodies (~100 kDa)^{18,19}, liposomes (~150 kDa)²⁰⁻²², and AAV vectors (~25 nm)²³⁻²⁶ (FIG. 1D, FIG. 3, FIG. 4). Over 200 patients have now been treated in Phase 1 clinical trials using FUS-BBB opening for conditions such as Alzheimer's disease, amyotrophic lateral sclerosis, brain tumors and Parkinson's disease (e.g., Clinical Trials NCT03551249. NCT03671889, NCT03608553); in a Phase I trial FUS-BBB was used to deliver a chemotherapy drug to glioblastoma patients (Clinical Trial NCT03551249). FUS systems for use in humans are commercially available (e.g., ExAblate Neuro, available from InSightec).

Thus, the present methods can include systemic (e.g., intravascular) administration of a contrast agent, e.g., clinically approved microbubbles, Microbubbles are typically 1-3 μm in diameter and have a poorly water-soluble gas core stabilized by shell-forming agents at the surface. A variety of microbubble shell compositions are clinically approved, including polymers, proteins, and lipids. See, e g., Hughes et al., Ultrasound Med Biol. 2023 July; 49(7):1479-1490, In some embodiments, the microbubble composition is a perflutren lipid microsphere suspension (e.g., Definity®). Following systemic (e.g., IV) administration of the microbubble contrast agent, focused ultrasound is directed to the desired portion of the brain.

Exemplary FUS-BBB methods include those described herein and in U.S. Pat. No. 6,514,221; Hughes et al., Ultrasound Med Biol. 2023 July; 49(7):1479-1490; Chang et al., J Korean Neurosurg Soc. 2023 March, 66(2): 172-182; Burgess et al., Expert Rev Neurother. 2015 May; 15(5): 477-491: Lipsman et al., Nature Communications 9:2336 (2018); and references 15-26 below.

In embodiments, the ultrasound carrier frequency is from 200 kHz to 1.5 MHz, e.g., 200 kHz to 1.0 MHz, 200 kHz to 500 kHz, 500 kHz to 1.5 MHz, 500 kHz to 1.0 MHz, or 1.0 MHz to 1.5 MHz. In embodiments, the burst duration of the ultrasound frequency is 10 ms or about 10 ms. In embodiments, the number of bursts is 30 to 180, e.g., 30 to 150, 30 to 100, 30 to 50, 50 to 180, 50 to 150, 50 to 100, 100 to 180, 100 to 150, or 150 to 180. In embodiments, the burst repetition frequency is from 0.25 to 2 Hz, e.g., 0.25 to 1.5 Hz, 0.25 to 1 Hz, 0.25 to 0.5 Hz, 0.5 to 2 Hz, 0.5 to 1.5 Hz, 0.5 to 1.0 Hz, 1 to 2 Hz, 1 to 1.5 Hz, or 1.5 to 2 Hz. In embodiments, the FUS Pressure is from 0.25 to 0.5 MPa.

### Engineered AAV Capsids

The present methods include administering engineered AAV capsids with improved BBB penetrance to deliver gene therapy reagents. Variants of AAV capsids have been developed using various methods, including directed evolution and rational design approaches, for improved BBB penetrance. One widely adopted approach to AAV capsid engineering is directed evolution, where capsids are selected out of a library of randomly generated candidates for a particular tissue tropism. Using this approach, novel neurotropic AAV capsids have been reported that outperform the “gold-standard” capsid AAV9 in mice. However, their CNS-transducing efficiencies in non-human primates are still low, raising doubts about their translatability into clinical application. Capsids AAV.CPP.16 (also referred to herein as CPP.16) and AAV.CPP.21 (also referred to herein as CPP.21) were developed using rational design, where capsids are modified using hypothesis-driven, mechanism-focused strategies. Capsid AAV.CPP.16 is 5-10 times more efficient than AAV9 in transducing the brain in non-human primates after systemic IV administration⁷². CPP.16 and CPP.21 are described below and in WO2020/014471, WO2021/142300, and WO2022/272240.

AAV is a tiny non-enveloped virus having a 25 nm capsid. No disease is known or has been shown to be associated with the wild-type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6, AV6.2, AAV7, AAV8, AAV9, rh.10, rh.39, rh.43 or CSp3: for CNS use, in some embodiments the AAV is AAV1, AAV2, AAV4, AAV5, AAV6, AAV8, or AAV9. As one example, AAV9 has been shown to somewhat efficiently cross the blood-brain barrier. Using the present methods, the AAV capsid can be genetically engineered to increase permeation across the BBB, or into a specific tissue, by insertion of a targeting sequence as described herein into the capsid protein, e.g., into the AAV9 capsid protein VP1 between amino acids 588 and 589.

An exemplary wild type AAV9 capsid protein VP1 (Q6JC40-1) sequence is as follows:

```
(SEQ ID NO: 1)
                10 20 30 40
MAADGYLPDW LEDNLSEGIR EWWALKPGAP QPKANQQHQD
                50 60 70 80
NARGLVLPGY KYLGPGNGLD KGEPVNAADA AALEHDKAYD
                90 100 110 120
QQLKAGDNPY LKYNHADAEF QERLKEDTSF GGNLGRAVEQ
              130 140 150 160
AKKRLLEPLG LVEEAAKTAP GKKRPVEQSP QEPDSSAGIG
              170 180 190 200
KSGAQPAKKR LNFGQTGDTE SVPDPQPIGE PPAAPSGVGS
              210 220 230 240
LTMASGGGAP VADNNEGADG VGSSSGNWHC DSQWLGDRVI
              250 260 270 280
TTSTRTWALP TYNNHLYKQI SNSTSGGSSN DNAYFGYSTP
              290 300 310 320
WGYFDENRFH CHESPRDWQR LINNNWGFRP KRLNFKLFNI
              360 330 340 350
QLPYVLGSAH QVKEVTDNNG VKTIANNLTS TVQVFTDSDY
              370 380 390 400
EGCLPPFPAD VEMIPQYGYL TLNDGSQAVG RSSFYCLEYF
              410 420 430 440
PSQMLRTGNN FQFSYEFENV PFHSSYAHSQ SLDRLMNPLI
              450 460 470 480
DQYLYYLSKT INGSGQNQQT LKFSVAGPSN MAVQGRNYIP
              490 500 510 520
GPSYRQQRVS TTVTQNNNSE FAWPGASSWA LNGRNSLMNP
              530 540 550 560
GPAMASHKEG EDRFFPLSGS LIFGKQGTGR DNVDADKVMI
              570 580 590 600
TNEEEIKTTN PVATESYGQV ATNHQSAQAQ AQTGWVQNQG
              610 620 630 640
ILPGMVWQDR DVYLQGPIWA KIPHTDGNFH PSPLMGGFGM
              650 660 670 680
KHPPPQILIK NTPVPADPPT AFNKDKLNSF ITQYSTGQVS
              690 700 710 720
VEIEWELQKE NSKRWNPEIQ YTSNYYKSNN VEFAVNTEGV
              730
YSEPRPIGTR YLTRNL
```

Thus provided herein are methods that use AAV that include one or more of the targeting peptide sequences described herein, e.g., an AAV comprising a capsid protein comprising a targeting sequence described herein, e.g., a capsid protein comprising SEQ ID NO:1 wherein a targeting peptide sequence has been inserted into the sequence. e.g., between amino acids 588 and 589. The following Table 1 provides exemplary targeting peptide sequences: the capsids can comprise a sequence from Table 1, or at least four, five, or six contiguous amino acids from Table 1.

TABLE 1
Exemplary Targeting Peptide Sequences

| Targeting peptide | Sequence | SEQ ID NO: |
|---|---|---|
| AAV.CPP.16 | TVSALK | 2 |
| AAV.CPP.21 | TVSALFK | 3 |
| AAV.CPP.16 retro-inverso | KLASVT | 4 |
| AAV.CPP.21 retro-inverso | KFLASVT | 5 |

In some embodiments, the AAV also includes a transgene sequence (i.e., a heterologous sequence), e.g., a transgene encoding a therapeutic agent, e.g., as described herein or as known in the art, or a reporter protein, e.g., a fluorescent protein, an enzyme that catalyzes a reaction yielding a detectable product, or a cell surface antigen. The transgene is preferably operably linked to sequences that promote/drive expression of the transgene in the target tissue.

Exemplary transgenes for use as therapeutics include mucolipin TRP cation channel 1 (MCOLN1); neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC), aspartoacylase (ASPA); colony stimulating factors (CSF); interleukins, such as IL-1. IL-2. IL-3. IL-4, IL-5, IL-6, IL-7, IL-8, IL-9, etc.: growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble gamma/delta T cell receptors, ligand-binding fragments of a soluble receptor, and the like; enzymes, such as α-glucosidase, imiglucarase, β-glucocerebrosidase, and alglucerase; enzyme activators, such as tissue plasminogen activator; chemokines, such as IP-10, monokine induced by interferon-gamma (Mig), Groa/IL-8, RANTES, MIP-1α, MIP-1B, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like: anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; relaxin; glial fibrillary acidic protein; follicle stimulating hormone (FSH); leukemia inhibitory factor (LIF); transforming growth factors (TGFs): tissue factors, luteinizing hormone; macrophage activating factors; tumor necrosis factor (TNF); neutrophil chemotactic factor (NCF); nerve growth factor; tissue inhibitors of metalloproteinases; IL-1 receptor antagonists; and the like. Some other examples of protein of interest include ciliary neurotrophic factor (CNTF); neurotrophins 3 and 4/5 (NT-3 and 4/5): glial cell derived neurotrophic factor (GDNF): aromatic amino acid decarboxylase (AADC): lysosomal acid lipase; phenylalanine hydroxylase (PAH): glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

The transgene can also encode an antibody, e.g., an immune checkpoint inhibitory antibody, e.g., to PD-L1, PD-1, CTLA-4 (Cytotoxic T-Lymphocyte-Associated Protein-4; CD152); LAG-3 (Lymphocyte Activation Gene 3; CD223); TIM-3 (T-cell Immunoglobulin domain and Mucin domain 3; HAVCR2): TIGIT (T-cell Immunoreceptor with Ig and ITIM domains); B7-H3 (CD276); VSIR (V-set immunoregulatory receptor, aka VISTA, B7H5, C10orf54); BTLA 30 (B- and T-Lymphocyte Attenuator, CD272); GARP (Glycoprotein A Repetitions; Predominant: PVRIG (PVR related immunoglobulin domain containing); or VTCN1 (Vset domain containing T cell activation inhibitor 1, aka B7-H4).

Other transgenes can include small or inhibitory nucleic acids that alter/reduce expression of a target gene, e.g., siRNA, shRNA, miRNA, antisense oligos, or long non-coding RNAs that alter gene expression (see, e.g., WO2012087983 and US20140142160), or CRISPR Cas9/cas12a and guide RNAs.

The virus can also include one or more sequences that promote expression of a transgene, e.g., one or more promoter sequences; enhancer sequences, e.g., 5′ untranslated region (UTR) or a 3′ UTR; a polyadenylation site; and/or insulator sequences, operably linked to the transgene. In some embodiments, the promoter is a brain tissue specific promoter, e.g., a neuron-specific or glia-specific promoter. In certain embodiments, the promoter is a promoter of a gene selected to from: neuronal nuclei (NeuN), glial fibrillary acidic protein (GFAP), MeCP2, adenomatous polyposis coli (APC), ionized calcium-binding adapter molecule 1 (Iba-1), synapsin I (SYN), calcium/calmodulin-dependent protein kinase II, tubulin alpha I, neuron-specific enolase and platelet-derived growth factor beta chain. In some embodiments, the promoter is a pan-cell type promoter, e.g., cytomegalovirus (CMV), CMV immediate enhancer/chicken β-actin hybrid (CAG), chicken β-actin (CBA), beta glucuronidase (GUSB), ubiquitin C (UBC), or Rous sarcoma virus (RSV) promoter. The woodchuck hepatitis virus posttranscriptional response element (WPRE) can also be used.

In some embodiments, the AAV also has one or more additional mutations that increase delivery to the target tissue, e.g., the CNS, or that reduce off-tissue targeting. e.g., mutations that decrease liver delivery when CNS, heart, or muscle delivery is intended (e.g., as described in Pulicherla et al. (2011) Mol Ther 19:1070-1078); or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience) 1-9, available at sfn.org/~/media/SfN/Documents/Short%20Courses/2011%20Short%20Course%201/2011_SC1_Grav.ashx.

### METHODS OF USE

The methods and compositions described herein can be used to deliver any composition, e.g., a sequence of interest to a tissue, e.g., to the brain, dorsal root ganglion or spinal cord (central nervous system), or heart or muscle (peripheral nervous system). In some embodiments, the methods include delivery to specific brain regions, e.g., cortex, cerebellum, hippocampus, substantia nigra, amygdala. In some embodiments, the methods include delivery to neurons, astrocytes, glial cells, or cardiomyocytes.

In some embodiments, the methods and compositions, e.g., AAVs, are used to deliver a nucleic acid sequence to a subject who has a disease, e.g., a disease of the CNS: see, e.g., U.S. Pat. Nos. 9,102,949; 9,585,971; and US20170166926. In some embodiments, the subject has a condition listed in Table 2; in some embodiments, the vectors are used to deliver a therapeutic agent listed in Table 2 for treating the corresponding disease listed in Table 2. The therapeutic agent can be delivered via a AAV vector as described herein, wherein the nucleic acid encodes a therapeutic protein or other nucleic acid such as an antisense oligo, siRNA, shRNA, and so on; or as a fusion protein/complex with a targeting peptide as described herein.

TABLE 2
Diseases

| Examples of diseases | Tissue targeted | Therapeutic agent |
|---|---|---|
| Parkinson's disease | CNS | GDNF, AADC |
| Alzheimer's disease | CNS | Tau antibody, APP |
| | | antibody |
| Huntington's disease | CNS | miRNA targeting HTT |
| Amyotrophic lateral sclerosis | CNS | shRNA targeting SOD |
| Multiple sclerosis | CNS | IFN-beta |
| Epilepsy | CNS | Neuropeptide Y |
| Stroke | CNS | IGF-1, osteopontin |
| Brain cancer | CNS | HSV.TK1, PD-1/PD- |
| | | L1 antibody |
| Spinocerebellar ataxia | CNS | RNAi targeting ataxin |
| Canavan disease | CNS | ASPA |
| Mucolipidosis IV | CNS | MCOLN1 |
| Metachromatic leukodystrophy | Nervous systems | ARSA, PSAP |
| Spinal muscular atrophy | Neuromuscular system | SMN1 |
| Friedreich's ataxia | Nervous systems, heart | Frataxin |
| X-linked myotubular | Neuromuscular system | MTM1 |
| myopathy | | |
| Pompe disease | Lysosome (global | GAA |
| | including CNS) | |
| Barth syndrome | Heart, muscle | TAZ |
| Duchenne muscular dystrophy | Muscle | dystrophin |
| Wilson's disease | Brain, liver | ATP7B |
| Crigler-Najjar syndrome type 1 | Liver | UGT1A1 |

In some embodiments, the compositions and methods are used to treat brain cancer. Brain cancers include gliomas (e.g., glioblastoma multiforme (GBM)), metastases (e.g., from lung, breast, melanoma, or colon cancer), meningiomas, pituitary adenomas, and acoustic neuromas. The compositions include a targeting peptide linked to an anticancer agent, e.g., a “suicide gene” that induces apoptosis in a target cell (e.g., HSV.TK1, Cytosine Deaminase (CD) from Herpes simplex virus or Escherichia coli, or Escherichia coli purine nucleoside phosphorylase (PNP)/fludarabine: see Krohne et al., Hepatology. 2001 September; 34(3):511-8: Dey and Evans, “Suicide Gene Therapy by Herpes Simplex Virus-1 Thymidine Kinase (HSV-TK)” (2011) DOI: 10.5772/18544) an immune checkpoint inhibitory antibody as known in the art or described herein. For example, an AAV vector comprising a targeting peptide as described herein can be used to deliver the “suicide gene” HSV.TK1 to a brain tumor. HSV.TK1 turns the otherwise “dormant” ganciclovir into a tumor-killing drug. Thus the methods can include systemically, e.g., intravenously, administering an AAV (e.g., AAV9) comprising a targeting peptide as described herein and encoding HSV.TK1, and the pro-drug ganciclovir, to a subject who has been diagnosed with brain cancer.

In some embodiments, the AAV is delivered at a dose of 2×10¹² or 4×10¹² or 2×10¹³ vg/kg. As 63% of clinical trials using systemic administration use a dose of 1.4×10¹² to 1.4×10¹⁴ vg/kg, this dose range is on the low end, providing the potential for cost savings (given the expense associated with producing AAV) and increased safety (as lower doses have bene associated with decreased side effects). See Au et al., “Gene Therapy Advances: A Meta-Analysis of AAV Usage in Clinical Settings,” Front. Med. (Lausanne) 8:809118 (2021).

### Pharmaceutical Compositions and Methods of Administration

The methods described herein include the use of pharmaceutical compositions comprising the engineered AAV as an active ingredient, and optionally comprising microbubbles (e.g., as described herein).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intraarterial, subcutaneous, intraperitoneal intramuscular or injection or infusion administration. Delivery can thus be systemic or localized.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration. For example, the compositions comprising an AAV comprising an engineered capsid with a targeting peptide as described herein and a composition comprising microbubbles can be provided together in a kit.

### EXAMPLES

The invention is further described in the following examples, which do not limit the scope of the invention described in the claims.

### Methods and Materials

The following methods and materials were used in the Examples below.

### FUS Blood-Brain Barrier Opening and AAV CPP Delivery

Mice were anaesthetized with a combination of ketamine and xylazine (K=80/X=10 mg/kg). The scalp fur was removed with an electric clipper and depilatory cream was applied to ensure that all the fur was removed to enable sufficient ultrasound coupling. A catheter was inserted into the tail vein for the administration of microbubbles, AAV and the MRI contrast agent gadolinium. The animal's head was fixed in a custom-made holder with ear bars for head immobilization and target referencing which was slotted into an in-house FUS system with the mice in supine position. The FUS system consists of a single element focused ultrasound transducer (4 cm diameter, 3.5 cm radius of curvature) operating at 690 kHz, three-axis manual positioning system and a passive cavitation detector. The head of the animal was coupled to the transducer with degassed and deionized water.

The sonications were applied using a function generator (33220A, Agilent), amplifier (240 L, E&I) and a customized MATLAB program which in turn drove the 690 kHz FUS transducer. FUS-mediated BBB opening was targeted to the right striatum and the parameters used were 10 ms bursts applied at 1 Hz repetition frequency for 120 s at 0.34 MPa⁷⁵. Two sonications were applied sequentially to the striatum based on previously established co-ordinates⁷⁶. The FUS sonications were performed immediately after the intravenous administration of microbubbles (20 μL/kg bolus injection of Definity). The AAV was injected immediately after sonications via the tail-vein catheter at 2×10¹¹ vector genomes (vg)/mouse (approximately 8×10¹² vg/kg based on 25 g mouse body weight).

### AAV Vector Production.

AAV was produced using standard triple transfection method. A series of quality control procedures were applied, including standardized viral titer measurement and infectivity testing, before AAVs were released for the in vivo mouse study. For delivery validation studies, we used a published AAV vector expressing nuclear red fluorescent protein (H2B-RFP) under the control of the ubiquitous promoter EF1a or CAG.

### Histology and Tissue Analysis.

Mice were sacrificed at three week post-treatment and their brains harvested. 30 μm coronal sections covering the FUS-targeted area were double stained with immunofluorescence for nuclear RFP and NeuN (neurons), Iba1 (microglia), and s100b and GFAP (astrocytes). Sections were imaged at 20× on an Olympus VS120 slide scanner microscope. RFP signal were quantified over striatal and cortical ROIs in both hemispheres; NeuN+RFP images were used to calculate percent of neurons transduced; Iba1 and GFAP images will be used to assess extent of microgliosis and astrocytosis.

### Example 1

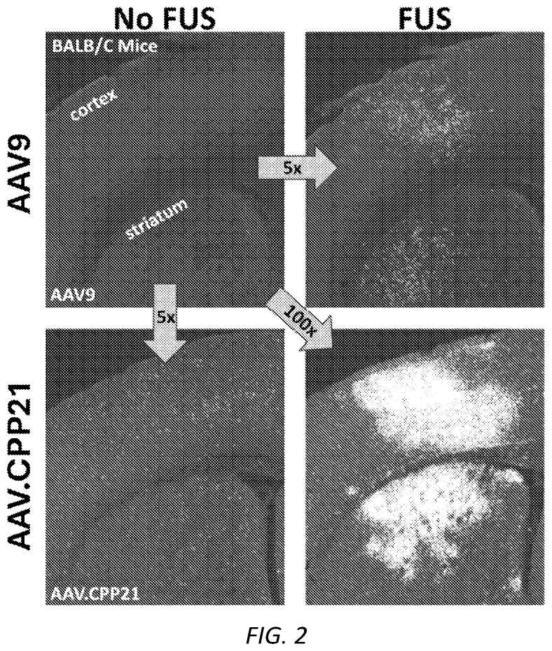

This example investigated the possibility of combing FUS-BBB opening with two AAV capsids, AAV.CPP.16 and AAV.CPP.21. Experiments were performed in N=3 mice per group with FUS targeted to one hemisphere. Two very promising results emerged relative to FUS+AAV9 delivery. Delivery of the RFP reporter gene in non-FUS targeted areas was substantially greater for both capsid variants than what was seen using either AAV9 or AAV.PHPeB, which was previously suggested to be able to provide improved delivery to the brain over AAV9 (FIG. 1). The improvement was far greater than expected. While transduction efficiency was improved by about 5-fold over AAV9 alone by either addition of the engineered capsid CPP.21 or the use of FUS-BBB, the improvement over AAV9 by the combination of using the engineered capsid CPP.21 with FUS-BBB was about 100 fold, an order of magnitude greater than would have been expected, as shown in FIG. 2. Thus, delivery in the FUS-targeted areas far exceeded expectations.

Neuronal transduction was evaluated in mice using FUS+AAV9, FUS+AAV.PHPeB, FUS+AAV.CPP.16, and FUS+AAV.CPP.21 (8e12 vg/kg AAVs or 2e11 vg per mouse). There was substantial expressing in neurons in the animals treated with FUS+AAV. CPP.16, and FUS+AAV CPP.21 (FIG. 3A). Quantification of percent neurons transfected in FUS-targeted (ultrasound) and non-targeted (control) hemispheres for cortex and striatum showed that neuronal transfection levels reached over 60% in cortex and over 40% in striatum in the animals treated with FUS+AAV.CPP.16, and FUS+AAV.CPP.21: the best results in the other conditions were less than about 10% in the cortex in AAV9-transduced animals (FIG. 3B). Although overall levels were low, astrocytes were also transduced at rates 5-10 fold higher with CPP.16 and CPP.21 than with AAV9 or AAV.PHPeB.

A dose-response study was conducted to determine whether even further reductions in dose could be used. As shown in FIGS. 4A-B, significant transduction and expression of the RFP transgene was seen when AAV.CPP.16 was delivered (with FUS) at 2×10\hat{ }13 vg/kg, 4×10 vg/kg, and 2×10 vg/kg, with levels over 80% of all cells with the 2×10\hat{ }13 vg/kg and 4×10 vg/kg doses.

### REFERENCES

1. Kulkami, J. A. et al. The current landscape of nucleic acid therapeutics. Nature Nanotechnology 16, (2021).
2. D. M., S. et al. AmVXS-101 phase 1 gene therapy clinical trial in SMA type 1: Experience with pre-existing anti-AAV9 antibody in the SMA1 population. Neurology (2017).
3. Pardridge, W. M. Blood-brain barrier delivery. Drug Discovery Today (2007). doi:10.1016/j.drudis.2006.10.013
4. Manfredsson, F. P., Rising, A. C. & Mandel. R. J. AAV9: A potential blood-brain barrier buster. Molecular Therapy (2009). doi:10.1038/mt.2009.15
5. Foust, K. D. et al. Intravascular AAV9 preferentially targets neonatal neurons and adult astrocytes. Nat. Biotechnol. (2009). doi:10.1038/nbt. 1515
6. Hinderer, C. et al. Severe Toxicity in Nonhuman Primates and Piglets Following High-Dose Intravenous Administration of an Adeno-Associated Virus Vector Expressing Human SMN. Hum. Gene Ther. (2018). doi:10.1089/hum.2018.015
7. Evers, M. M. et al. AAV5-miHTT Gene Therapy Demonstrates Broad Distribution and Strong Human Mutant Huntingtin Lowering in a Huntington's Disease Minipig Model. Mol. Ther. (2018). doi:10.1016/j.ymthe.2018.06.021
8. Samaranch, L. et al. MR-guided parenchymal delivery of adeno-associated viral vector serotype 5 in non-human primate brain. Gene Ther. (2017). doi:10.1038/gt.2017.14
9. Chen, X. et al. Engineered AAVs for non-invasive gene delivery to rodent and non-human primate nervous systems. Neuron 110, (2022).
10. Chan, K. Y. et al. Engineered AAVs for efficient noninvasive gene delivery to the central and peripheral nervous systems. Nat. Neurosci. 20, (2017).
11. Challis, R. C. et al. Systemic AAV vectors for widespread and targeted gene delivery in rodents. Nat. Protoc. 14, (2019).
12. Hordeaux, J. et al. The Neurotropic Properties of AAV-PHP.B Are Limited to C57BL/6J Mice. Molecular Therapy (2018). doi:10.1016/j.ymthe.2018.01.018
13. Hynynen, K., McDannold, N., Vykhodtseva, N. & Jolesz, F. A. Noninvasive MR imaging-guided focal opening of the blood-brain barrier in rabbits. Radiology 220, 640-6 (2001).
14. Park. J., Zhang, Y., Vykhodtseva. N., Jolesz. F. A. & McDannold, N. J. The kinetics of blood brain barrier permeability and targeted doxorubicin delivery into brain induced by focused ultrasound. J. Control. Release 162, 134-42 (2012).
15. Arval, M., Vykhodtseva. N., Zhang, Y. Z., Park, J. & McDannold, N. Multiple treatments with liposomal doxorubicin and ultrasound-induced disruption of blood-tumor and blood-brain barriers improve outcomes in a rat glioma model. J. Control. Release (2013). doi:10.1016/j.jconrel.2013.04.007
16. Jung, B., Huh, H., Lee. E. hee, Han, M. & Park, J. An advanced focused ultrasound protocol improves the blood-brain barrier permeability and doxorubicin delivery into the rat brain. J. Control. Release (2019). doi:10.1016/j.jconrel.2019.10.044
17. McDannold, N. et al. Acoustic feedback enables safe and reliable carboplatin delivery across the blood-brain barrier with a clinical focused ultrasound system and improves survival in a rat glioma model. Theranostics (2019). doi:10.7150/thno.35892
18. Jordao. J. F. et al. Antibodies targeted to the brain with image-guided focused ultrasound reduces amyloid-β plaque load in the TgCRND8 mouse model of Alzheimer's disease. PLoS One (2010). doi:10.1371/journal.pone.0010549
19. Kinoshita, M., McDannold, N., Jolesz, F. A. & Hvnynen, K. Targeted delivery of antibodies through the blood-brain barrier by MRI-guided focused ultrasound. Biochem. Biophys. Res. Commun. (2006). doi:10.1016/j.bbrc.2005.12.112
20. Aryal, M. et al. MRI Monitoring and Quantification of Ultrasound-Mediated Delivery of Liposomes Dually Labeled with Gadolinium and Fluorophore through the Blood-Brain Barrier. Ultrasound Med. Biol. (2019).doi:10.1016/j.ultrasmedbio.2019.02.024
21. Treat, L. H., McDannold, N., Zhang, Y., Vykhodtseva, N. & Hynynen, K. Improved Anti-Tumor Effect of Liposomal Doxorubicin After Targeted Blood-Brain Barrier Disruption by MRI-Guided Focused Ultrasound in Rat Glioma. Ultrasound Med. Biol. (2012). doi:10.1016/j.ultrasmedbio.2012.04.015
22. Yang, F. Y. et al. Focused ultrasound and interleukin-4 receptor-targeted liposomal doxorubicin for enhanced targeted drug delivery and antitumor effect in glioblastoma multiforme. J Control. Release (2012). doi:10.1016/j.jconrel.2012.02.023
23. Alonso, A. et al. Focal delivery of AAV2/1-transgenes into the rat brain by localized ultrasound-induced BBB Opening. Ann. Neurosci. 21, 22 (2014).
24. Wang, S., Konofagou, E. E., Olumolade, O. O., Sun, T. & Samiotaki. G. Noninvasive, neuron-specific gene therapy can be facilitated by focused ultrasound and recombinant adeno-associated virus. Gene Ther. 22, 104-110 (2014).
25. Weber-Adrian, D. et al. Strategy to enhance transgene expression in proximity of amyloid plaques in a mouse model of Alzheimer's disease. Theranostics (2019). doi:10.7150/thno.36718
26. Thevenot, E. et al. Targeted Delivery of Self-Complementary Adeno-Associated Virus Serotype 9 to the Brain, Using Magnetic Resonance Imaging-Guided Focused Ultrasound. Hum. Gene Ther. 23, 1144-1155 (2012).
27. Yao. Y. et al. Variants of the adeno-associated virus serotype 9 with enhanced penetration of the blood-brain barrier in rodents and primates. Nat. Biomed. Eng. (2022). doi:10.1038/s41551-022-00938-7
28. Future Market Insights. Gene Therapy in CNS Disorder Market Snapshot (2023 to 2033). (2023).
29. Precedence Research. Central Nervous System Therapeutic Market. (2023).
30. Grand View Research. Huntington's Disease Treatment Market Size, Share & Trends Analysis Report By Treatment. (2023).
31. Thevenot, E. et al. Targeted delivery of self-complementary adeno-associated virus serotype 9 to the brain, using magnetic resonance imaging-guided focused ultrasound. Hum. Gene Ther. (2012). doi:10.1089/hum.2012.013
32. Kofoed, R. H. et al. Transgene distribution and immune response after ultrasound delivery of rAAV9 and PHP.B to the brain in a mouse model of amyloidosis. Mol. Ther.—Methods Clin. Dev. 23, (2021).
33. Li, H., Heath, J. E., Trippett, J. S., Shapiro, M. G. & Szablowski, J. O. Engineering viral vectors for acoustically targeted gene delivery. bioRxiv (2021).
34. Todd, N. et al. Focused ultrasound induced opening of the blood-brain barrier disrupts inter-hemispheric resting state functional connectivity in the rat brain. Neuroimage 178, 414-422 (2018).
35. Todd, N., Zhang, Y., Livingstone, M., Borsook, D. & McDannold, N. The neurovascular response is attenuated by focused ultrasound-mediated disruption of the blood-brain barrier. Neuroimage (2019). doi:10.1016/j.neuroimage.2019.116010
36. Todd, N. et al. Modulation of brain function by targeted delivery of GABA through the disrupted blood-brain barrier. Neuroimage (2019). doi:10.1016/j.neuroimage.2019.01.037
37. Hwu, P. W. et al. Gene therapy in the putamen for curing AADC deficiency and Parkinson's disease. EMBO Mol. Med. 13, (2021).
38. Weissgerber, T. L., Garcia-Valencia, O., Garovic, V. D., Milic, N. M. & Winham, S. J. Why we need to report more than ‘data were analyzed by t-tests or ANOVA’. Elife (2018). doi:10.7554/eLife.36163

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of delivering a transgene to a cell in the brain of a mammalian subject, the method comprising:
using focused ultrasound-mediated disruption of the blood-brain barrier (FUS-BBB opening) in a selected region of the brain; and
systemically administering to the subject an adeno-associated virus (AAV) comprising (i) a capsid protein comprising an amino acid sequence that comprises at least four contiguous amino acids from the sequence TVSALK (SEQ ID NO:2); TVSALFK (SEQ ID NO:3); KLASVT (SEQ ID NO:4); or KFLASVT (SEQ ID NO:5), and (ii) a transgene.

2. The method of claim 1, wherein the amino acid sequence comprises at least five contiguous amino acids from the sequence TVSALK (SEQ ID NO:4); TVSALFK (SEQ ID NO:8); KLASVT (SEQ ID NO:83); or KFLASVT (SEQ ID NO:84).

3. The method of claim 1, wherein the amino acid sequence comprises at least six contiguous amino acids from the sequence TVSALK (SEQ ID NO:4); TVSALFK (SEQ ID NO:8); KLASVT (SEQ ID NO:83); or KFLASVT (SEQ ID NO:84).

4. The method of claim 1, wherein using FUS-BBB comprises:
systemically administering a microbubble contrast agent, and
applying focused ultrasound energy to the blood-brain barrier (FUS-BBB) in the selected region of the brain.

5. The method of claim 4, wherein the microbubble contrast agent comprises perflutren lipid microspheres.

6. The method of claim 4, wherein the focused ultrasound energy is applied at a frequency of from 200 kHz to 1.5 MHz, a burst duration of 10 ms, a number of bursts of from 30 to 180, a burst repetition frequency of from 0.25 to 2 Hz and a FUS Pressure of from 0.25 to 0.5 MPa.

7. The method of claim 1, wherein the subject is a human subject.

8. The method of claim 1, wherein the AAV is AAV9.

9. The method of claim 8, wherein the AAV9 comprises AAV9 VP1.

10. The method of claim 9, wherein the targeting sequence is inserted in a position corresponding to amino acids 588 and 589 of AAV9 VP1 comprising SEQ ID NO:85.

11. The method of claim 7, wherein the AAV is administered by parenteral delivery.

12. The method of claim 11, wherein the parenteral delivery is via intravenous delivery.

13. The method of claim 4, wherein the microbubble contrast agent and the AAV are delivered by intravenous delivery, optionally in a single composition.

14. The method of claim 1, wherein the transgene is listed in Table 2, optionally selected from the group consisting of mucolipin TRP cation channel 1 (MCOLN1); neuronal apoptosis inhibitory protein (NAIP), nerve growth factor (NGF), glial-derived growth factor (GDNF), brain-derived growth factor (BDNF), ciliary neurotrophic factor (CNTF), tyrosine hydroxlase (TH), GTP-cyclohydrolase (GTPCH), amino acid decorboxylase (AADC), and aspartoacylase (ASPA); RNA interference; gene editing machineries.

15. The method of claim 14, wherein the transgene is linked to sequences that promote expression of the transgene.

16.-30. (canceled)


SEQUENCE LISTING (5 sequences)

SEQ ID NO: 1 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 6 aa, synthetic construct
TVSALK

SEQ ID NO: 3 - protein, 7 aa, synthetic construct
TVSALFK

SEQ ID NO: 4 - protein, 6 aa, synthetic construct
KLASVT

SEQ ID NO: 5 - protein, 7 aa, synthetic construct
KFLASVT